(19) United States
(12) Patent Application Publication
(10) Pub. No.: US 2006/0031969 A1
(43) Pub. Date: Feb. 9, 2006

(54) Hybrid bluegrass plants and seeds

(75) Inventors: John R. Hardison, Corvallis, OR (US); Jay B. Burr, Salem, OR (US)

Correspondence Address: JONES DAY, 77 WEST WACKER, CHICAGO, IL, 60601-1692, US

(73) Assignee: OMS Investments, Inc., Los Angeles, CA (US)

(21) Appl. No.: 11/197,808

(22) Filed: Aug. 5, 2005

Related U.S. Application Data
(60) Provisional application No. 60/599,446, filed on Aug. 6, 2004.

(51) Int. Cl. A01H 5/00

(52) U.S. Cl. 800/320

(57) ABSTRACT

The present invention relates to methods of breeding Poa arachnifera and Poa pratensis hybrid plants, the Poa arachnifera and Poa pratensis hybrid plants per se, as well as the plant parts, including the seeds. The present invention further relates to Poa arachnifera and Poa pratensis hybrid seeds exhibiting reduced, ranging down to no, cotton webbiness. Methods of using the hybrid plants and the hybrid seeds are also provided. The hybrid plants are particularly suitable for use in lawns, golf courses, sod, and other turfs.

### CROSS-REFERENCE TO RELATED APPLICATION

This application claims priority to Provisional Application No. 60/599,446, filed Aug. 6, 2004, which is incorporated by reference herein in its entirety.

### 1. FIELD OF THE INVENTION

The present invention relates to methods of breeding Poa arachnifera and Poa pratensis hybrid plants, the Poa arachnifera and Poa pratensis hybrid plants per se, as well as the plant parts, including the seeds. The present invention further relates to Poa arachnifera and Poa pratensis hybrid seeds. Methods of using the hybrid plants and the hybrid seeds are also provided. The hybrid plants are particularly suitable for use in lawns, golf courses, sod, other turfs and forage.

### 2. BACKGROUND OF THE INVENTION

Poa arachnifera Torr., Texas bluegrass, is a dioecious species with individual plants having either male or female flowers (i.e. staminate or pistillate flowers). Through genetic recombination, the progeny of the Poa arachnifera are heterozygous and heterogeneous. There are also basic morphological differences between panicles of male and female Poa arachnifera plants (other than the obvious absence of male or female reproductive parts).

Poa pratensis L., Kentucky bluegrass, has perfect flowers (both male and female reproductive organs in the same flower) and is considered to be a facultative apomict. In other words, most seed derived progeny of the Kentucky bluegrasses are predominantly asexual clones of the mother plant. In general, Poa pratensis cultivars are selected to have a high level of apomixis (asexual seed development), which ensures high levels of homogeneity among seed derived plants (plants are heterozygous) from the cultivar. In apomixis of Poa pratensis, plants develop macrospores (egg cell, 1 n genetic component) and microspores (pollen, 1 n genetic component). However, the macrospore generally disintegrates prior to fertilization by a pollen grain. Tissues within the ovary of a somatic (2 n) nature develop into the embryo. Pollen fertilization is necessary, but predominantly only leads to development of the endosperm, which does not result in a genetic influence on plant morphological development and therefore, does not impact either genotype or phenotype of the resulting plant. The 2 n (maternal) derivation of the cell(s) leading to the development of the embryo within the seed ensures predominant homogeneity among the progeny. Therefore, the measure of apomixis is an estimate of the asexual reproduction versus sexual recombination within a generation of Poa pratensis.

Heat and drought tolerance are important traits for lawns, commercial grounds and golf course roughs. Texas bluegrass (Poa arachnifera Torr.) is a heat and drought tolerant bluegrass from the south-central United States. However, due to its poor turf qualities and seed production characteristics, it has not been developed for commercial use. Kentucky bluegrass (Poa pratensis L.) is a popular, widely utilized cool-season grass. While Kentucky bluegrass is the primary turfgrass of choice for northern lawns, its poor heat tolerance has prevented adoption by homeowners in border and southern states.

Prior work on hybridization of Texas and Kentucky bluegrasses has been shown to dramatically increase the heat tolerance of progeny that have Kentucky bluegrass characteristics. In the hybridization of Texas and Kentucky bluegrasses, the Texas bluegrass parent plants were selected for their seed yielding potential, while the Kentucky bluegrass parent plants were selected based on superior turfgrass characteristics. One example of a Texas and Kentucky bluegrass hybrid plant is the Kentucky bluegrass variety “Reveille” (Reg. no. CV-53. PI 603946). It was originally named TXKY 16-1. Reveille is an F₁ hybrid between Texas and Kentucky bluegrasses. In spite of the superior heat tolerance, Reveille has not experienced commercial success due to difficulties in seed production, in particular cotton webbiness on the seeds. Hitchcock describes Texas bluegrass, Poa arachnifera, as ‘ . . . conspicuously cobwebby, . . . , copiously webby at base, . . . ”, Read (2001) states ‘One undesirable trait of Texas bluegrass is the dense woolly-pubescence with long kinky hairs attached at the base of the lemmas that make seed harvesting and handling very difficult.” (International Turfgrass Society Research Journal 9: p. 202). Due to these problems, Reveille is typically sodded as the method of planting, as seeding is impractical due to the cotton on the seeds.

Seed production of Poa species is generally handled differently than other turfgrass species. Poa pratensis L. is swathed at a lower moisture percentage than other species (Poa trivialis is more prone to seed shatter and therefore swathed at a higher moisture percentage). This low moisture percentage may be due in part to the presence of varying amounts of cotton on the seed which leads to a lesser susceptibility of shattering. In general, this species is harder to combine than other turfgrass species in that the inflorescence requires a more rigorous threshing process. Spikelets within an inflorescence may clump together due to the cotton on the lemma of individual disseminules (seeds). The severity of the threshing process of seeds in a machine called a combine is increased for bluegrass since the seeds need to be removed from the spikelets and separated from one another in order to be retained in the combine. Materials that do not fall through a specified size screen flow through and out the back of the combine. While the screen sizes within a combine are not so small as to exclude material that is somewhat larger than a single seed, they have to be small enough to remove most of the straw and chaff. Bluegrass seed has varying amounts of fine hair attached to the seed called “cotton”. Clumping due to excessive cotton in the spikelets of an inflorescence can result in seeds sticking together, forming a clump that is too large to fall through the screens, and therefore, the seeds are discharged out of the back of the combine.

Poa species, when they are taken into the cleaning plant, are generally put through a machine called a ‘debearder’. The function of this machine is to further separate bluegrass seeds as there are quite often ‘doubles’ in the post-combined seed. ‘A double’ is two seeds still joined by the interseed portion of the rachilla of the spikelet. The presence of cotton on the seed at this point can negatively influence the effectiveness of the debearder in that seed held together by the cotton is harder to separate, and more likely to be retained without falling through the screen.

The retained seeds are then ‘cleaned’ as the final process in seed production. The seeds are again run over screens in order to remove both coarse materials (e.g. stems) and fine materials (e.g. soil particles). Air is also used to blow away light materials (e.g. portions of dried leaves still within the sample).

Therefore a reduction in cotton is beneficial in all three of the generally employed seed harvesting and cleaning steps in processing Poa species. Increased presence of cotton on grass seeds leads to greater difficulty in the seed harvesting process, increased seed loss, which leads to greater cost being incurred in order to retain and clean the seed. It could also lead to greater damage of the seed as the rigor of processing must be increased in order to separate and recover the seed. Accordingly, an advantage of the present invention is the provision of a method for selecting for hybrid plants with reduced cotton webbiness of the seeds.

### 3. BRIEF SUMMARY OF THE INVENTION

The invention is based on the discovery by the applicants that by hybridizing Poa arachnifera selected for turfgrass characteristics with Poa pratensis selected for seed yield characteristics, many of the F₁ population of hybrid plants exhibited reduced, ranging down to no, cotton webbiness on the seeds. The selection of parent species for the above described characteristics would have been counterintuitive because Poa pratensis generally exhibits superior turfgrass qualities which would logically be the selection criteria for that parent species.

The resulting Poa arachnifera and Poa pratensis hybrid plant and hybrid seed have an advantage over known hybrids in that it does not present the difficulties in harvesting and handling of the seeds resulting from cotton webbiness and therefore the Poa arachnifera and Poa pratensis hybrid plant can be efficiently seeded as a method of planting. In preferred embodiments, the Poa arachnifera is the female plant. In another preferred embodiment, the Poa pratensis is the female plant. Not to be bound by any mechanism, cotton (webbiness) is related to reduced soil:seed:water contact which increases the time for seedling emergence. Thus, a reduction in the cotton webbiness of the seeds enhances seedling vigor through rapid emergence.

According to the invention, there is provided a method of breeding Poa arachnifera and Poa pratensis to produce a Poa arachnifera and Poa pratensis hybrid. plant (F₁) that is different from all known varieties of hybrid bluegrass. Also provided is a novel Poa arachnifera and Poa pratensis hybrid plant exhibiting reduced, ranging down to no, cotton (webbing) on the hybrid seeds. In a preferred embodiment, the Poa arachnifera is a Poa arachnifera Torr. In another preferred embodiment, the Poa pratensis is Poa pratensis L. In further preferred embodiment, the Poa arachnifera is a female plant. In another preferred embodiment, the Poa pratensis is a female plant.

In preferred embodiments, the present invention provides a method of breeding Poa arachnifera Torr. and Poa pratensis L. to produce a Poa arachnifera Torr. and Poa pratensis L. hybrid plant (F₁). Also provided is a novel Poa arachnifera Torr. and Poa pratensis L. hybrid plant exhibiting reduced, ranging down to no, cotton (webbing) on the seed. In particular, the present invention further relates to Poa arachnifera Torr. and Poa pratensis L. hybrid seeds that exhibit reduced, ranging down to no, cotton webbiness. In a particular embodiment, the novel Poa arachnifera Torr. and Poa pratensis L. hybrid plant further exhibits a high level of apomixis. For the purpose of the present invention, the Poa arachnifera Torr. may be a male or a female plant and the Poa pratensis L. may be a male or a female plant. In a specific embodiment, a Poa arachnifera Torr. female plant is crossed with a Poa pratensis L. male plant. In another specific embodiment, a Poa pratensis L. female plant is crossed with a Poa arachnifera Torr. male plant.

In a specific embodiment, the invention encompasses hybrid plants exhibiting reduced, ranging down to no, cotton (webbing) on the seeds and a high level of apomixis produced by the method of backcrossing Poa arachnifera and Poa pratensis hybrid plants to parent Poa arachnifera plants or other sexually compatible Poa species. Methods of producing such hybrid backcrossed plants are also encompassed by the invention.

In specific embodiments, the invention provides for methods of breeding Poa arachnifera Torr. and Poa pratensis L. hybrid plants. In one embodiment, the invention provides for a method of producing a Poa arachnifera Torr. and Poa pratensis L. hybrid plant, comprising hybridizing Poa arachnifera Torr. and Poa pratensis L. parent plants and selecting for a progeny with seeds exhibiting reduced, ranging down to no, cotton (webbiness). In certain embodiments, the F₁ plant is backcrossed to one of the parent plants to produce a backcrossed hybrid plant. In another embodiment, an F₂ plant selected for high levels of apomixis and/or commercially acceptable turfgrass quality characteristics are backcrossed to one of the parent plants to produce a backcrossed hybrid plant. In certain embodiments, the F₁ plant is backcrossed to a different variety of one of the parent plant to produce a backcrossed hybrid plant. In another embodiment, the F₂ plant selected for high levels of apomixis and/or commercially acceptable turfgrass quality characteristics are backcrossed to a different variety of one of the parent plants to produce a backcrossed hybrid plant. In certain embodiments, the Poa arachnifera Torr. parent plant is selected based on desirable turfgrass characteristics and the Poa pratensis L. parent plant is selected for based on desirable seed yield characteristic. In certain embodiments, the plant is from an intercross between a Poa arachnifera Torr. and a Poa pratensis L. hybrid plants. In specific embodiments, the plant resulting from the cross between the selected Poa arachnifera Torr. and Poa pratensis L. is crossed to a Poa arachnifera Torr. and Poa pratensis L. hybrid plant.

The invention also provides for hybrid plants and hybrid seeds produced by hybridizing Poa arachnifera Torr. and Poa pratensis L., wherein the seeds exhibit reduced, ranging down to no, cotton (webbing) and a high level of apomixis. In other embodiments, the hybrid plants exhibit low level of apomixis. In preferred embodiments, the hybrid plants, their progeny and plant parts including seeds can be produced by the methods described herein.

The invention is also directed to a vegetative sprig or a clone of a Poa arachnifera and Poa pratensis hybrid grass plant wherein the seeds of such a plant exhibits reduced, ranging down to no, cotton (webbing) and a high level of apomixis. In one embodiment, the invention provides for a Poa arachnifera and Poa pratensis hybrid grass plant wherein the seeds of such a plant exhibit reduced, ranging down to no, cotton (webbing) and a high level of apomixis, or its parts. The parts include leaves, stems, roots, nodes, rhizomes, tillers, seeds, pollen, ovules, embryos, root tips, anthers, hairs, lemmas, spikelets, florets, flowers, stalks and tissue cultures thereof. The invention also provides for protoplasts produced from such regenerable cells. In certain embodiments, cells of the tissue culture are derived from a tissue which includes, but are not limited to, leaves, pollens, embryos, roots, root tips, nodes, rhizomes, stems, tillers, anthers, pollens, hairs, lemmas, spikelets, florets, flowers, and stalks. In other embodiments, the invention provides for a Poa arachnifera and Poa pratensis hybrid plant wherein the seeds of such a plant exhibit reduced, ranging down to no, cotton (webbing) and/or a high level of apomixis wherein said hybrid plant is regenerated from tissue culture. In preferred embodiments, the Poa arachnifera is Poa arachnifera Torr. In preferred embodiments, the Poa pratensis is Poa pratensis L. In a preferred embodiment, representative seeds of said plant were deposited on Jul. 22, 2005 under American Type Culture Collection (ATCC) No. ______ as the ‘HB 129’ variety. The invention also provides for an essentially homogeneous population of Poa arachnifera Torr. and Poa pratensis L. hybrid plants produced by growing the seed of the ‘HB 129’ variety. The invention also provides for a Poa arachnifera Torr. and Poa pratensis L. hybrid plant expressing all the physiological and morphological characteristics of the of the ‘HB 129’ variety. In a preferred embodiment, the Poa arachnifera Torr. is the female plant.

The invention provides for an F₁ population of Poa arachnifera and Poa pratensis hybrid plants wherein various percentages (at least 1%, 2%, 5%, 10%, 20%, 30%, 40%, 50%, 60%) of plants produce seeds having reduced, ranging down to no, cotton webbiness. The invention provides for an F₁ population of Poa arachnifera and Poa pratensis hybrid plants said population comprising a greater number of plants that produce seeds that exhibit reduced, ranging down to no, cotton webbiness in comparison to known F₁ populations of Poa arachnifera and Poa pratensis hybrid plants.

The invention also provides for a method of producing grass seeds, comprising planting the seed for a Poa arachnifera and Poa pratensis hybrid plant under conditions that result in the germination of the seed, growth of grass plants and setting of progeny seed and harvesting of the progeny seed. In certain embodiments, the Poa arachnifera and Poa pratensis hybrid plant produces or is grown from seeds that exhibit reduced, ranging down to no, cotton (webbing). In a related embodiment, the hybrid plants further exhibit a high level of apomixis.

The invention provides for a mixture of grass seeds comprising the grass seed of a Poa arachnifera and Poa pratensis hybrid plant wherein the seeds of such a plant exhibit reduced, ranging down to no, cotton (webbing).

The invention also provides for kits for growing grass. The kit comprises one or more packages of the seeds of the invention together with instructions for planting such seeds in or as a hay stand, a pasture stand, a roadside or right-of way, a sod, a golf course fairway, a golf course rough, a lawn, an athletic field, a park, or a forage stand. In certain embodiments, the packages further comprise seeds of other plant species.

The invention also provides for methods for planting seeds in an area lacking Poa arachnifera and Poa pratensis hybrid plants wherein the seeds are derived from Poa arachnifera and Poa pratensis L. hybrid plants exhibiting reduced, ranging down to no, cotton (webbing) on the seeds and the seeds are planted in or as a hay stand, a pasture stand, a roadside or right-of way, a sod, a golf course fairway, a golf course rough, a lawn, an athletic field, a park, or a forage stand. In a related embodiment, the novel Poa arachnifera and Poa pratensis hybrid plant further exhibits a high level of apomixis.

In another embodiment, this invention is directed to a hybrid plant derived from crossing Poa arachnifera and Poa pratensis grasses as plant parents, the hybrid plant or its seed exhibiting at least one characteristic including: a reduced amount of cotton webbing on a seed of the hybrid plant; a reduced cotton webbing density on a seed of the hybrid plant; a reduced length of cotton webbing as a percentage of lemma length on a seed of the hybrid plant; increased panicle openness; or reduced panicle compactness.

In a specific embodiment, the amount of cotton webbing on the seed of the hybrid plant; the cotton webbing density on the seed of the hybrid plant; the length of cotton webbing as a percentage of lemma length on the seed of the hybrid plant; and the panicle compactness, are reduced in comparison with Reveille. In a specific embodiment, the panicle openness is increased in comparison with Reveille.

In a further specific embodiment, one of the plant parents is Poa arachnifera Torr. In other specific embodiment, the Poa arachnifera Torr. is a female plant. In a preferred embodiment, one of the plant parent is Poa pratensis L. In a preferred embodiment, the plant parents are Poa arachnifera Torr. and Poa pratensis L. In a specific embodiment, the amount of cotton webbing on a seed of the hybrid plant is rated 2 or below, on a scale of 1 to 5. In a specific embodiment, the cotton webbing density on a seed of the hybrid plant is rated 2.6 or below, on a scale of 1 to 5. More preferably, the cotton webbing density on a seed of the hybrid plant is rated 2 or below, on a scale of 1 to 5. In a specific embodiment, the length of cotton webbing as a percentage of lemma length is less than about 75%. In a specific embodiment, the panicle openness is in a range of about 30% to about 100%. In a specific embodiment, the panicle compactness is less than about 5%. In a specific embodiment, the reduced amount of cotton webbing on a seed is determined after conditioning of the seed.

In a specific embodiment, the hybrid plant is derived from backcrossing to a Poa arachnifera. In another specific embodiments, the hybrid plant is derived from backcrossing to a Poa pratensis. In a further specific embodiment, the hybrid plant is derived from intercrossing to a Poa arachnifera and a Poa pratensis hybrid plant.

The present invention is also directed to the progeny of a hybrid plant derived from crossing Poa arachnifera and Poa pratensis grasses as plant parents, the progeny of the hybrid plant or its seed exhibiting at least one characteristic including: a reduced amount of cotton webbing on a seed of the progeny of the hybrid plant; a reduced cotton webbing density on a seed of the progeny of the hybrid plant; a reduced length of cotton webbing as a percentage of lemma length on a seed of the progeny of the hybrid plant; increased panicle openness of the progeny of the hybrid plant; or reduced panicle compactness of the progeny of the hybrid plant. In another specific embodiment, the progeny of the hybrid plant may not have any of the above characteristics. The invention is also directed to a seed of the hybrid plant, a seed of the progeny of the hybrid plant, a seed mixture of the hybrid plant or a seed mixture of the progeny of the hybrid plant.

In specific embodiments, the progeny of the hybrid plant is derived from backcrossing to a Poa arachnifera. In other specific embodiment, the progeny of the hybrid plant is derived from backcrossing to a Poa pratensis. In other specific embodiment, the progeny of the hybrid plant is derived from intercrossing to a Poa arachnifera and a Poa pratensis hybrid plant.

The invention is also directed to a method of producing a seed of the hybrid plant comprising: planting the seed of the invention under conditions that result in the germination of the seed, growth of the hybrid plant, setting of a progeny seed; and harvesting of the progeny seed.

The invention is also directed to a method of producing progeny of the hybrid plant of the present invention comprising: crossing the Poa arachnifera and Poa pratensis; harvesting the seed; and germinating the seed to produce at least one progeny of the hybrid plant.

The invention is directed to a method of producing a progeny of the hybrid plant of the present invention comprising the steps of: selecting a Poa arachnifera and a Poa pratensis grass plant; crossing the plants; harvesting and growing the seed resulting from the crossing; selecting F₁ plants grown from the seed; harvesting seed from the F₁ plants; selecting seed with reduced amount of webbing; and germinating the seed to produce the hybrid plant. In a preferred embodiment, the F₁ plant comprises perfect flowers, wherein the perfect flowers having both male and female reproductive parts on individual florets. In preferred embodiments, the method further comprises the step of selecting for F₂ plants with a rapid germination time. In other preferred embodiments, the method firther comprises the step of growing and selecting for high levels of apomixis and/or commercially acceptable turfgrass quality characteristics. In a preferred embodiment, the Poa arachnifera is a female plant. In another preferred embodiment, the Poa pratensis is a female plant. In a preferred embodiment, the Poa arachnifera is selected for a superior turfgrass characteristic. In another preferred embodiment, the Poa pratensis is selected for a superior seed yield characteristic.

### 3.1. DEFINITIONS

The terms “Poa arachnifera Torr. and Poa pratensis L. hybrid plant”, “Poa arachnifera and Poa pratensis hybrid plant”, “Poa arachnifera X Poa pratensis hybrid plant”, “Hybrid plant”, “Texas X Kentucky bluegrass hybrid” and any other comparable references mean: a plant derived from cross-pollinating Poa arachnifera and Poa pratensis; the progeny of such a plant by sexual reproduction by crossing to sexually compatible Poa species to produce e.g., F₂, F₃, F₄ or F₅ generation plants etc.; the progeny of a backcross of such a plant or its progeny to one or more parent species; the progeny from asexual reproduction from the seeds; or a hybrid plant derived from cloning. In preferred embodiment, the Poa arachnifera is Poa arachnifera Torr. In specific embodiment, the Poa pratensis is Poa pratensis L.

The term “hybrid seed” encompasses the seed of a hybrid plant.

The terms “cotton webbing” “cotton webbiness”, “webbiness”, and “cotton of seeds” mean hair fibers produced on the external parts of grass seeds that form a dense woolly pubescence of long kinky hairs attached at the base of the lemmas that make harvesting and handling very difficult.

The term “apomixis” means the production of seeds and vegetative propagules by asexual methods through specialized generative tissues that does not include the fusion of egg and sperm cells, such as seeds.

The term “seed conditioning” means converting raw seeds to finished seeds acceptable for sale and use. In specific embodiments, the seed conditioning is a cleaning process involving a debearder, screens, scalpers, air tables, shakers, rotary indent, and vibrators. The field run seeds harvested by a combine may be reduced by 5-30%. Light weight, immature seeds are discarded as well as pieces of stems, dirt, and weed seed. The conditioned seeds are then ready for testing and sale.

### 4. DESCRIPTION OF THE DRAWINGS

FIG. 1 is a separated bluegrass spikelet showing “very high density of long webby cotton”, rating 5, on lemmas (actual length of separated spikelet=17 mm).

FIG. 2 is a separated bluegrass spikelet showing “high density of medium-long webby cotton”, rating 4, on lemmas (actual length of separated spikelet=12 mm).

FIG. 3 is a separated bluegrass spikelet showing “medium density of medium length webby cotton”, rating 3, on lemmas (actual length of separated spikelet=8.5 mm).

FIG. 4 is a separated bluegrass spikelet showing “low density of short webby cotton”, rating 2, on lemmas (actual length of separated spikelet=10 mm).

FIGS. 5A-5D are the ratings of webby cotton density within separated spikelets from bluegrass (Poa pratensis, Poa arachnifera, and Poa pratensis X Poa arachnifera). (A) Rating 5=very high density of long webby cotton; (B) Rating 4=high density of medium-long webby cotton; (C) Rating 3=medium density of medium length webby cotton; (D) Rating 2=Low density of short webby cotton; Rating 1=No cotton webbing (not shown).

FIG. 6A is an illustration of a poa spikelet.

FIG. 6B is an illustration of a poa floret.

FIGS. 7A-C is an illustration of (A) HB129 panicle; (B) Reveille panicle; and (C) Texas Bluegrass panicle.

### 5. DETAILED DESCRIPTION OF THE INVENTION

The present invention is based, in part, on the inventors' discovery that selection and breeding of certain parent plants produced Poa arachnifera and Poa pratensis hybrid plants with seed characterized by reduced, ranging down to no, cotton webbiness and/or high level of apomixis. Accordingly, the present invention relates to methods of breeding such hybrid plants, the resulting hybrid plants, progeny and clones of such hybrid plants, plant parts, and culture of plant parts.

### 5.1. Poa arachnifera And Poa pratensis Hybrid Plant And Seed Characteristics

The Poa arachnifera and Poa pratensis hybrid plants and plant parts of the invention, comprise a novel combination of traits, including reduced, ranging down to no, cotton (webbing) on the hybrid seeds and a high level of apomixis. In other embodiments, low level of apomixis is preferred. The Poa arachnifera and Poa pratensis hybrid plants and hybrid seeds of the invention may comprise other novel combinations of characteristics including, but not limited to, heat tolerance and resistance against certain diseases. In specific embodiments, the Poa arachnifera and Poa pratensis hybrid plants and the hybrid seeds of the invention, may have a unique combination of advantageous traits as exemplified by hybrid variety ‘HB 129’0 (see U.S. Plant patent, filed on even date herewith).

In a preferred embodiment, Poa arachnifera and Poa pratensis hybrid plants and hybrid seeds of the invention have a high level of seedling vigor and rapid stand establishment, high sod strength, and a relatively high level of competition with Poa annua in the seedling year when compared to Kentucky bluegrass varieties. In more preferred embodiments, the Poa arachnifera is Poa arachnifera Torr. and the Poa pratensis is Poa pratensis L.

### 5.1.1. Apomixis Level

In general, the range of apomixis levels of Poa pratensis is from about 85% to 95% or more. Reveille has an apomixis level of about 90%. The apomixis level of Poa arachnifera and Poa pratensis hybrid plants and hybrid seeds of the invention is greater than that of known Poa arachnifera and Poa pratensis hybrids. In one embodiment, the apomixis level of Poa arachnifera and Poa pratensis hybrid plants and hybrid seeds of the invention is approximately 85% to 90%, 90% to 95%, 95% to 100% of the total seed. In preferred embodiments, the level of apomixis in a population of Poa arachnifera and Poa pratensis hybrid plants produced by the methods of the invention is approximately 95%, 96%, 97%, 98%, 99%, or 100% of the total seed. In one embodiment, the level of apomixis in a population of Poa arachnifera and Poa pratensis hybrid plants produced by the methods of the invention is approximately 95% of the total seed. The apomixis level may be affected by the vegetative characteristics of a plant such as plant density.

### 5.1.2. Seed Characteristics

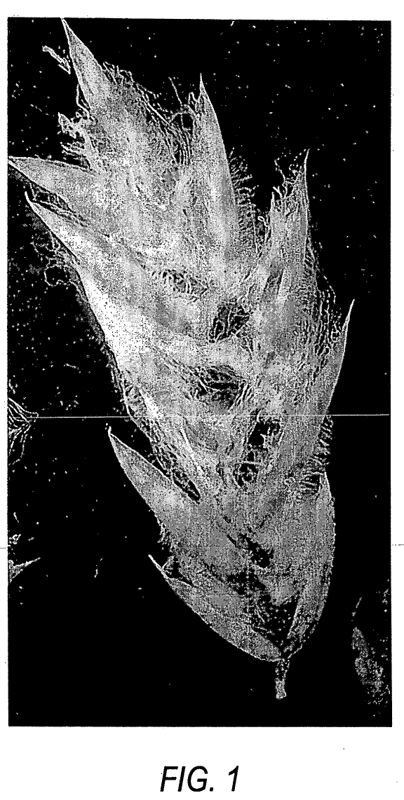

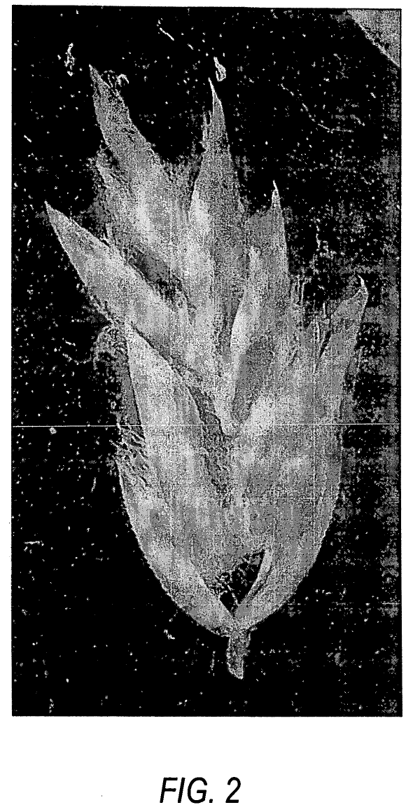

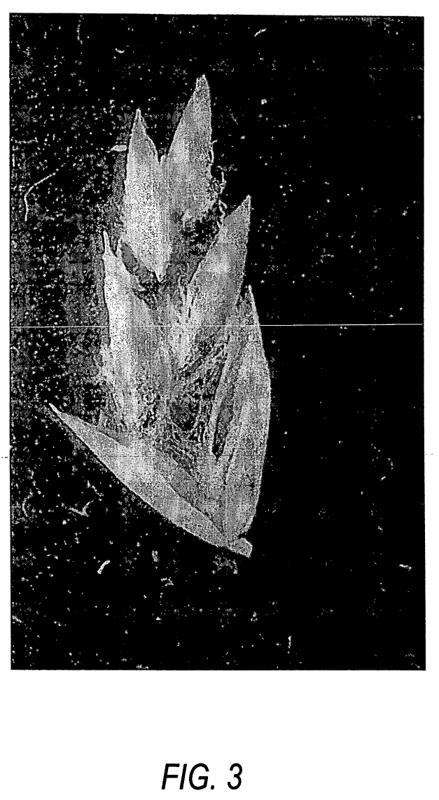

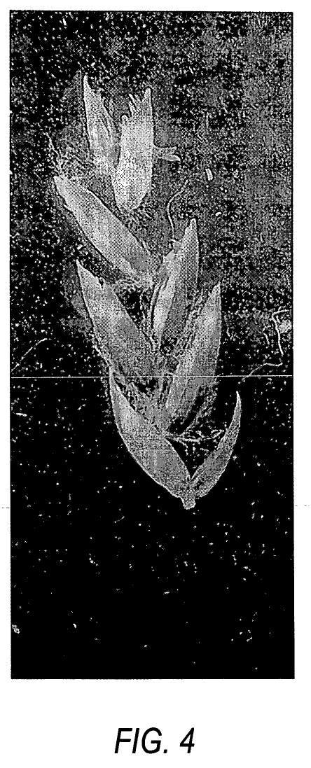

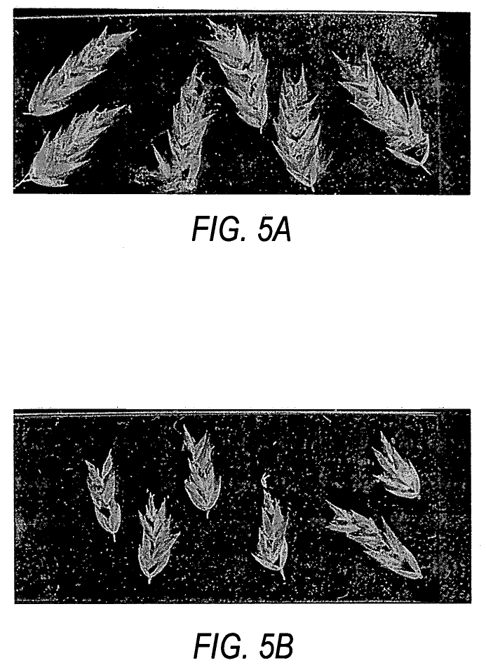

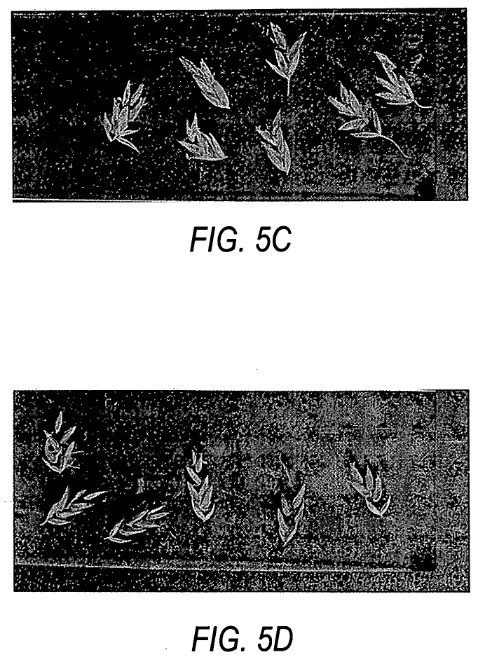

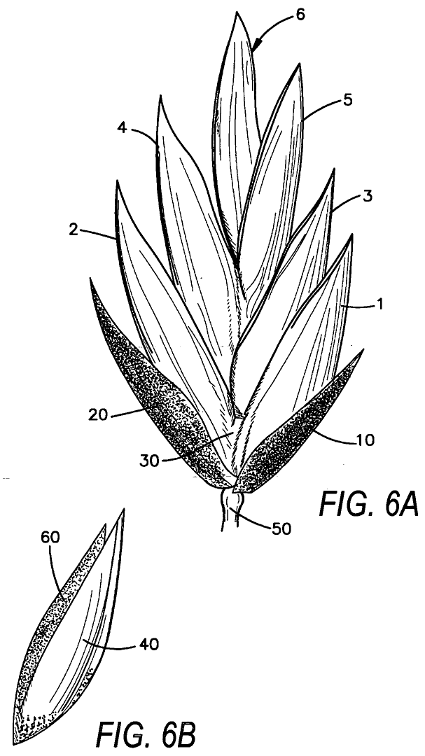

The seeds of the hybrid plants of the present invention exhibit reduced, ranging down to no, cotton webbing or webbiness. The amount of webby cotton may be measured with a scale of 1 to 5 as shown in FIGS. 1-5. FIG. 1 shows very high density of long webby cotton, rating 5; FIG. 2 shows a high density of medium-long webby cotton, rating 4; FIG. 3 shows a medium density of medium length webby cotton, rating 3; FIG. 4 shows a low density of short webby cotton, rating 2; FIG. 5 shows a comparison of various amounts of webby cotton within separated spikelets from bluegrass. FIG. 6A illustrates a poa spikelet. This spikelet comprises 6 lemmas that are floret-bearing leaves or florets, 1, 2, 3, 4, 5, and 6; two glumes, 10, 20, that are non-floret bearing leaves, a rachilla, 30, that is the jointed axis of the spikelet where the florets are attached, a rachis, 50, that is the spine or backbone support of the spikelets. The central line of a compound inflorescence is called an axis. Poa has soft curly hairs on the lower part of the lemma mid-nerve and marginal nerves and white cottony hairs at the base of the lemma. FIG. 6B illustrates a Poa floret enclosed by a lemma, 40, and a palea, 60.

In certain embodiments, the webbiness of the seeds of the hybrid plants of the invention is reduced, ranging down to no, cotton in comparison to the webbiness of parent plant. In certain embodiments, the webbiness of the seeds of the hybrid plants of the invention is reduced, ranging down to no, cotton in comparison to the webbiness of seeds produced by F₁ plants which parents were not selected. In a related embodiment, the webbiness of the seeds of hybrid plants of the invention is reduced, ranging down to no, cotton in comparison to the webbiness of known Poa arachnifera and Poa pratensis hybrid plants. In preferred embodiments, the hybrid plant or its seed exhibiting at least one characteristic including: a reduced amount of cotton webbing on the seed of the hybrid plant; a reduced cotton webbing density on the seed of the hybrid plant; a reduced length of cotton webbing as a percentage of lemma length on the seed of the hybrid plant; increased panicle openness; and reduced panicle compactness.

In certain embodiments, about 2%, 5%, 10%, 20%, 30%, 40%, 50%, 60% of the seeds of the F₁ hybrid plants produced by the methods of the invention have reduced, ranging down to no, cotton webbiness. In preferred embodiments, about 15% of the seeds of the F₁ hybrid plants produced by the methods of the invention have reduced, ranging down to no, cotton webbiness. In certain embodiments, about 70%, 80%, 90%, 95%, 99% or 100% of the seeds of the F₂ hybrid plants produced by the methods of the invention have reduced, ranging down to no, cotton webbiness. In certain related embodiments, the percentage of seeds exhibiting reduced, ranging down to no, cotton webbiness in the F₂ lines, i.e., the progeny of a single F₁ plant, is about 70%, 80%, 90%, 95%, 99% or 100%.

The morphological characteristics of the Poa arachnifera×Poa pratensis hybrid seeds of the invention are typically similar to those of Kentucky bluegrass (Poa pratensis L.) varieties.

### 5.2. Plant Breeding And Selection Methods

Numerous breeding and selection techniques can be used in the claimed methods for hybridizing selected Poa arachnifera plants and selected Poa pratensis plants to produce hybrid plants and hybrid seeds with desirable traits that can be reproduced with uniformity and stability. Hybrid plants can be further bred by both self-pollination and cross-pollination to improve uniformity and stability or to introduce desirable traits from varieties of Poa arachnifera or Poa pratensis. Suitable Poa arachnifera selections that are useful in the present invention include but are not limited to Tx 10-10, Tx 7-23 and Tx 6-17. Suitable Poa pratensis varieties that are useful in the present invention include but are not limited to Geronimo, Ascot, Abbey and Midnight. Suitable Poa species that are useful in the present invention include but are not limited to Poa interior and Poa palustris.

In preferred embodiments, one parent plant is selected from Poa arachnifera plants based on improved turf characteristics, including, but not limited to, increased tiller density, leaf color, and/or plant height, in comparison to typical cultivars and wild types of Poa arachnifera. Methods for measuring such characteristics are well known in the art. The other parent plants are selected from Poa pratensis plants based on increased seed production and/or increased inflorescence number, in comparison to typical cultivars and wild types of Poa pratensis. In a preferred embodiment, each parent plant breeds true and is highly uniform. In a preferred embodiment, the female parent is selected from Poa arachnifera plants. In another preferred embodiment, the male parent is selected from Poa arachnifera plants. In preferred embodiments, one or more of the parent plants is dioecious.

In an embodiment, the invention provides for a method of producing a Poa arachnifera and Poa pratensis hybrid plant, comprising the steps of a) selecting a Poa arachnifera plant and a Poa pratensis plant; b) crossing the Poa arachnifera plant with the Poa pratensis plant; c) harvesting and growing the seed resulting from the cross; d) selecting F₁ plants with perfect flowers grown from the seed of step c); e) harvesting seed from said perfect flowered F₁ plants; f) selecting seed with at least one characteristic including a reduced amount of cotton webbing on the seed; a reduced cotton webbing density on the seed; a reduced length of cotton webbing as a percentage of lemma length on the seed; increased panicle openness; or reduced panicle compactness; g) germinating the seed (progeny) of step f) and selecting for progeny with a rapid germination time; and h) growing and selecting for high levels of apomixis and/or commercially acceptable turfgrass quality characteristics.

In an embodiment breeding starts with the crossing of two parent genotypes, such as two species of the genus Poa, each of which may have one or more desirable characteristics that is lacking in the other or which complements the other. If the two original parents do not provide all the desired characteristics, other sources can be included in the breeding population. In this method, superior plants are self-pollinated (selfed) and selected in successive filial generations. In the succeeding filial generations the heterozygous condition gives way to homogeneous lines as a result of self-pollination and selection. Typically, five or more successive filial generations of self-pollination and selection is practiced. After a sufficient amount of inbreeding, successive filial generations will serve to increase the presence of the desirable traits.

Backcrossing can be used to improve a hybrid. Backcrossing can be used to transfer a specific desirable trait from a donor parent, to a recurrent parent which has overall desirable characteristics yet lacks that specific desirable trait (Poehlman et al., 1995; Fehr, 1987; Sprague and Dudley, 1988). The specific desirable trait can be transferred into a background by first crossing a recurrent parent and a donor parent (non-recurrent parent). The progeny are then mated back to the recurrent parent followed by selection in the resultant progeny for the desired trait to be transferred from the non-recurrent parent as well as selection for the characteristics of the recurrent parent. Backcrossing is repeated for several generations until a plant having the specific desirable trait and most or all of the advantageous traits of the recurrent parent is achieved. The number of backcross generations can be less if molecular markers are used during selection or parent plants with known genotypes are used as the donor parent. The last backcross generation is then selfed to give pure breeding progeny for the gene(s) controlling the desirable traits being transferred. Backcrossing can be combined or used subsequent or prior to other breeding strategies. In an embodiment, the selected parent plants of Poa arachnifera and Poa pratensis are crossed using Poa arachnifera as the female parent. The resulting F₁ or F₂ progeny selected for desirable traits can then be backcrossed to a different variety of Poa arachnifera or Poa pratensis having a desirable characteristic that is absent or different in the female parent. Examples of desirable traits include, but are not limited to, heat tolerance, or drought tolerance.

Recurrent selection can also be used in the methods of the invention. Recurrent selection involves individual plants cross pollinating with each other to form progeny which are then grown. The superior progeny are then selected by any number of methods, which include individual plant, half-sib progeny, full-sib progeny, selfed progeny and topcrossing. The elected progeny are cross pollinated with each other to form progeny for another population. This population is planted and again superior plants are selected to cross-pollinate with each other. Recurrent selection is a cyclical process and therefore can be repeated as many times as desired. The objective of recurrent selection is to improve the traits of a population. The improved population can then be used as a source of breeding material to obtain inbred lines to be used in hybrids or used as parents for a synthetic cultivar.

Mutation breeding can also be practiced with the methods of the invention. Examples of mutation breeding can be found in “Principals of Cultivar Development” Fehr, 1993 Macmillan Publishing Company the disclosure of which is incorporated herein by reference.

Molecular markers can be used in the plant breeding and selection methods of the invention and include, but are not limited to, Isozymes, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs), Simple Sequence Length Polymorphisms (SSLPs) (Williams et al., 1990), and Single Nucleotide Polymorphisms (SNPs) (Wang et al., 1998). For example, see Glick and Thompson, Methods In Plant Molecular Biology And Biotechnology 269-284 (CRC Press, Boca Raton, 1993). One use of molecular markers is Quantitative Trait Loci (QTL) mapping. QTL mapping is the use of markers, which are known to be closely linked to alleles that have measurable effects on a quantitative trait. Selection in the breeding process is based upon the accumulation of markers linked to the positive effecting alleles and/or the elimination of the markers linked to the negative effecting alleles from the plant's genome. QTL methodology is described in detail in Lynch and Walsh (1998, Genetics and Analysis of Quantitative Traits, Sinauer Associates, Inc., Sunderland Mass.). Multiple interval mapping may also be used to genotype individual plant and estimate heritabilities of quantitative traits (Kao et al. 1999 Genetics 152: 1203-1216). Specifically, linkage mapping in apomictic and sexual Poa pratensis L. genotyping may be used (Porceddu et al. 2002 Theor Appl Genet 104(2-3):273-280. See also, RAPD-based genetic relationships in Poa pratensis L. (Curley et al. 2004 Crop Sci. 44:1299-1306), which are incorporated by reference herein in their entireties.

In certain embodiments, the contribution of each parent to a hybrid plant or hybrid plant parts may be measured using molecular markers as described above by one skilled in the art. In specific embodiments, genetic divergence of a hybrid plant from Poa arachnifera is at least 1-2%, 2-5%, 5-10%, 10-20%, 20-30%, 30-40%, 40-50%, 50-60%, 60-70%, 70-80%, 80-90%, or 90-99%. In other specific embodiments, genetic divergence of a hybrid plant from Poa pratensis is at least 1-2%, 2-5%, 5-10%, 10-20%, 20-30%, 30-40%, 40-50%, 50-60%, 60-70%, 70-80%, 80-90%, or 90-99%. In preferred embodiments, genetic divergence of a hybrid plant from Poa arachnifera Torr. is at least 1-2%, 2-5%, 5-10%, 10-20%, 20-30%, 30-40%, 40-50%, 50-60%, 60-70%, 70-80%, 80-90%, or 90-99%. In other preferred embodiments, genetic divergence of a hybrid plant from Poa pratensis L. is at least 1-2%, 2-5%, 5-10%, 10-20%, 20-30%, 30-40%, 40-50%, 50-60%, 60-70%, 70-80%, 80-90%, or 90-99%. In preferred embodiments, the hybrid plant or its seed exhibiting at least one characteristic including: a reduced amount of cotton webbing on a seed of the hybrid plant; a reduced cotton webbing density on a seed of the hybrid plant; a reduced length of cotton webbing as a percentage of lemma length on a seed of the hybrid plant; increased panicle openness; and reduced panicle compactness.

In one embodiment of the breeding and selection methods of the invention, parent, F1, or F2 plants are selected for apomixis. It is known that an obligate or near obligate apomictic line will have less variation than a sexual line which results in increased uniformity of characteristics. Apomixes can increase and/or restore fertility of interspecifc hybrids (Read and Bashaw, 1969; and Bashaw and Hana, 1990). Thus, the breeding and selection methods of the invention can be practiced using statistical techniques designed to optimize selection for a desirable characteristics. Such statistical techniques are well known in the art.

Selection for rapid germination means selecting for seed that germinate in the shorter time periods, for example, selecting the first 1%, 2%, 3%, 4%, 5%, 10%, 15%, 20%, 30%, or 40% of seeds to germinate in a sample of seeds. Selection for rapid germination can also mean selecting the seeds that germinate in a certain time period such as about 3 days, 4 days, 5 days, 6 days, 7 days, 8 days, 9 days, 10 days, 11 days, 12 days, 13 days, 14 days, 15 days, 16 days, or 17 days. Selection for rapid germination can also mean selecting seeds that germinate in a shorter time period in comparison to known or parental varieties. In growing plants, plants can be grown by seed or by asexually propagation by rhizomes, tillers or disseminules. Plants can also be maintained, grown, and reproduced by tissue culture using techniques known in the art.

### 5.3. Uses of Poa arachnifera And Poa pratensis Hybrid Plants And Seeds

The Poa arachnifera and Poa pratensis hybrid plants and hybrid seeds of the present invention have a variety of uses that will be apparent to one skilled in the art including, but not limited to, turf grass breeding, breeding for forage grasses, seed production, and sod production. In certain embodiments, the hybrid plants and hybrid seeds of the present invention is in a hay stand, a pasture stand, a roadside or right-of way, a sod, a golf course fairway, a golf course rough, a lawn, an athletic field, a park, or a forage stand.

### 6. EXAMPLES

The following examples illustrate the breeding and selection of plants as well as establishment of turfs and emergence of the seedlings. These examples should not be construed as limiting.

### 6.1. Example 1

### Breeding And Selection of Poa arachnifera×Poa pratensis Hybrid ‘HB 129’

The ‘HB 129’ bluegrass variety is an example of a plant produced by the methods of the invention having been selected from the progeny of Texas bluegrass (Poa arachnifera Torr.) female plant 10-10 (seed parent), with cv. ‘Geronimo’ Kentucky bluegrass (pollen parent) (Poa pratensis L.).

### 6.2. Selection of Parent Plants

The seed parent plants were selected from Poa arachnifera Torr. plants based on improved turf characteristics, including, but not limited to, increased tiller density, leaf color, and plant height, in comparison to typical cultivars and wild types of Poa arachnifera Torr. Methods for measuring such characteristics are well known in the art. The pollen parent plants were selected from Poa pratensis L. plants based on increased seed production and increased inflorescence number, in comparison to typical cultivars and wild types of Poa pratensis L.

### 6.3. Breeding Methodology

Breeding methodology for Poa pratensis×Poa arachnifera comprises the following processes:
(a) Selection of parents: Parental selections have been made on the following basis: Poa arachnifera—female plants with good turfgrass quality characteristics were selected; Poa pratensis plants with good turfgrass quality and good seed production characteristics including reduced cotton were selected.
(b) Manipulation of flowering of both parental species. Since P. arachnifera tends to flower earlier than P. pratensis, the selected parents were isolated to reduce the risk of unwanted pollen (microspore) from fertilizing the female P. arachnifera macrospore. Manipulation may include the use of greenhouses or growth cabinets (increased temperatures and increased light durations) to speed up P. pratensis reproductive growth and the use of reduced light durations and reduced temperatures to impeded reproductive growth in P. arachnifera.
(c) Pollen receptor plants (Poa arachnifera female plants) were kept under good environmental conditions to reduce stress and increase seed set.
(d) Seed was harvested at maturity, stored under favorable conditions to allow for post harvest dormancy reduction. Imbibed seed was treated with reduced temperatures and light to enhance the breaking of post-harvest dormancy. The seed was germinated and seedlings transplanted into a spaced plant environment to maximize the potential for identification of desirable traits.
(e) Plants were allowed to undergo reproductive induction conditions and to flower. Flowers were studied to identify plants for perfect flowers (the presence of both male and female reproductive parts on individual florets), female flowers only, or male flowers only.
(f) Perfect flowered plants and female hybrid plants were identified with at least one characteristic including: a reduced amount of cotton webbing on a seed of the hybrid plant; a reduced cotton webbing density on a seed of the hybrid plant; a reduced length of cotton webbing as a percentage of lemma length on a seed of the hybrid plant; increased panicle openness; or reduced panicle compactness.
(g) Progeny were grown from perfect flowered plants and examined in comparison to the parental hybrid for trueness to type, and an estimation of apomixes potential. Plant morphology and seed characteristics are also examined.
(h) Promising lines were advanced to end use trials and seed production trials.
(i) Female plants selected in (f) above were examined via progeny for their potential in future breeding efforts.

### 6.4. Breeding And Selection

The selected Poa arachnifera Torr. female plants were pollinated with pollen from the selected Poa pratensis L. The seed resulting from the pollination was harvested and grown as an F₁ population and seed were harvested from F₁ plants with perfect flowers or female hybrid plants and screened for at least one characteristic including: a reduced amount of cotton webbing on a seed of the hybrid plant; a reduced cotton webbing density on a seed of the hybrid plant; a reduced length of cotton webbing as a percentage of lemma length on a seed of the hybrid plant; increased panicle openness; or reduced panicle compactness. Seeds that rapidly germinated were grown to maturity. From these mature plants, plants with high levels of apomixis and commercially acceptable turfgrass quality characteristics were selected for further propagation or breeding.

### 6.5. Characteristics of ‘HB 129’ Bluegrass

As a result of this breeding, a distinct variety, named ‘HB 129’ bluegrass, was produced and asexually propagated by rhizomes, tillers and disseminules. The highly apomictic seed of ‘HB 129’ bluegrass was produced first at Gervais, Ore. This seed was used to plant turf performance evaluation trials and later, seed production fields.

‘HB 129’ bluegrass is perennial with creeping rhizomes forming a dense turf. When plants overwinter in the field and grow undisturbed by clipping, culms are semi-erect averaging 64.8 cm in length. The vegetative leaf averages 4.5 mm in width. The flag leaf averages 6.65 cm in length, 3.55 mm in width, has a sheath length of 14.3 cm. The flag leaf averages more hairs on the ligule than other varieties. The panicle averages 10.8 cm in length. The lowest whorl of the panicle averages 4.4 branches.

For the vegetative leaf, the number of hairs is average for the leaf sheath margin, above average for the dorsal side of the leaf blade, above average for the upper margin of the ligule, and below average for the collar margin compared to other varieties. ‘HB 129’ differs from the Kentucky bluegrass varieties in regard to such morphological characteristics as seed length and width, culm length, and hairs on the collar margins of the vegetative leaf, on the upper surface of the vegetative leaf, on the ligule of the flag leaf and anther color. ‘HB 129’ produces inflorescences relatively early compared with Kentucky bluegrasses. ‘HB 129’ has performed well throughout the U.S. as exhibited by good turf quality ratings under reduced management inputs in comparison with other Kentucky bluegrass varieties and other Texas×Kentucky bluegrass hybrids. In addition, it has a light green color with good turf density which can be maintained throughout the growing season providing a rapid development of sod strength, and a medium to high seed yielding capacity. Further description of ‘HB 129’ bluegrass characteristics and comparisons to similar varieties and parent plants is found in the plant patent for ‘HB 129’ bluegrass filed on even date herewith, identified by attorney Docket No.: Hardison 1-11-1-1, which is incorporated by reference herein in its entirety.

‘HB 129’ has demonstrated relatively rapid germination and emergence compared to other hybrids including Reveille in both fall and spring sowings (Table 1). Table 1 shows germination rating comparison for HB 129 to hybrid bluegrasses and Kentucky bluegrasses planted in turfgrass plots at Cleveland, Tex. Data were collected from three replicates. ‘HB 129’ has shown greatly reduced seed cotton compared to Reveille. The presence of the cotton on the seeds of Reveille has been found to result in poor seed recovery which renders Reveille commercially unacceptable as a seeded variety.

TABLE 1

| | | Germination Rating | |
|---|---|---|---|
| | | | Spring |
| | | Fall establishment | establishment |
| Plant | | 15 days after seeding | 8 days after seeding |
| HB129 | Tx x Ky | 2.7 | 1.7 |
| Ascot | Ky | 2.7 | 2.7 |
| Abbey | Ky | 1.7 | 2.0 |
| Coventry | Ky | 1.7 | 1.3 |
| Reveille | Tx x Ky | 0.0 | 0.0 |
| | LSD (0.05) | 0.90 | 0.74 |

Tx x Ky - Texas x Kentucky bluegrass hybrid
Ky - Kentucky bluegrass
0 - no germinated seed visible
9 - all seed germinated and visible

The cotton webbiness on seeds of ‘HB 129’ is significantly reduced in comparison to other Poa arachnifera Torr.×Poa pratensis L. hybrid bluegrasses as indicated in Table 2. Table 2 is a comparison of HB 129 with other hybrid bluegrass for cotton on conditioned seed.

TABLE 2

| Plant Type | Amount of cotton webbing/cotton rating |
|---|---|
| HB 129 | Reduced or no cotton/rating 1 |
| HB 329 | Moderate level of cotton/rating 2 |
| Reveille | Copious amount of cotton/rating 3 |

Table 3 shows rating of the cotton webbing density within separated spikelets of unconditioned seeds (florets were pulled loose of spikelets to expose on each individual floret of specific spikelets); harvested from various bluegrass cultivars grown in greenhouse under controlled environment at Gervais, Ore. (average of six panicles and two spikelets per panicle and harvested from the lower whorl panicle branches). The rating corresponds to the scale in FIGS. 1-5. HB 129 and HB 130 both show a reduction in cotton webbiness density compared to Reveille hybrid bluegrass.

TABLE 3

| | Cotton Webbing Density (5 = Most and 1 = None) |
|---|---|
| Plant | (three replicates with 12 subsamples per replicate) |
| HB 129 | 2.0 |
| HB 130 | 2.0 |
| HB 329 | 3.5 |
| Reveille | 3.0 |
| Ascot | 2.8 |
| Texas 51-90 | 4.8 |
| Geronimo | 2.0 |
| LSD (P = 0.05) | 0.4 |
| CV | 8.6 |

Table 4 show rating of cotton webbing density within intact spikelets of unconditioned seeds (spikelets were removed from panicles, viewed and rated when intact); harvested from various bluegrass cultivars grown in greenhouse under controlled environment at Gervais, Ore. (average of 10 spikelets per panicle and harvested from the second whorl panicle branches).

TABLE 4

| | Average Cotton Webbing Density |
|---|---|
| | (5 = Most and 1 = None) |
| Plant | (from three replicates with 10 subsamples per replicate) |
| HB 129 | 2.00 |
| Texas 51-90 | 5.00 |
| Reveille | 3.33 |
| Ascot | 3.00 |
| Geronimo | 2.70 |
| LSD (P = 0.05) | 0.77 |
| CV | 12.76 |

Table 5 shows rating of cotton webbing density in panicles (panicles were viewed and rated intact); they were harvested from various bluegrass cultivars and grown in field nursery at Gervais, Ore. (average of 5 subsamples).

TABLE 5
3 replicates with 5 subsamples per replicate

| Plant | Cotton Webbing Density (5 = Most and 1 = None) |
|---|---|
| HB 129 | 2.0 |
| Texas 49-90 | 4.3 |
| Texas 39-88 | 3.8 |
| HB 342 | 3.7 |
| Reveille | 2.8 |
| Ascot | 2.4 |
| HB 130 | 2.3 |
| Geronimo | 2.1 |
| LSD (P = 0.05) | 0.744 |
| CV | 14.73 |

Table 6 shows lemma length (eyepiece assisted, 7×), lemma cotton length (% of lemma length), lemma cotton length calculation, length of the cotton (eyepiece assisted, 7×) and a visual rate of the density of the lemma cotton from spikelets harvested from various bluegrass cultivars grown in a greenhouse under controlled environment (average 4 florets subsamples-lower whorl branches) at Gervais, Ore. Lemmas were mounted on pinheads prior to rating. Data were collected from three replicates. HB 129, HB 130 and HB 329 all show reduced lemma cotton length (column C) when compared to Reveille hybrid bluegrass and Texas 51-90 Texas bluegrass.

TABLE 6
3 replicates with four subsamples per replicate

| | | | Lower Whorl | | |
|---|---|---|---|---|---|
| | Lower Whorl | Lemma Cotton | Lemma Cotton Length | Lower Whorl | Density of |
| | Lemma Length | Length (Visual)z | (Lemma Length × %) | Lemma Cotton Length | Lemma Cotton |
| | Eyepiece (7x) | Of Lemma Length | (A) × (B) = C | Eyepiece (7x) | Visual Rank |
| Column | A | B | C | D | E |
| Plant | mm | Percent | mm | mm | 5 = Most 1 = None |
| HB 129 | 3.9 | 49.1 | 1.9 | 1.8 | 2.0 |
| Texas 51-90 | 5.7 | 126.1 | 7.0 | 6.8 | 4.1 |
| Reveille | 4.4 | 94.2 | 4.1 | 4.1 | 3.1 |
| Ascot | 4.1 | 52.5 | 2.2 | 2.2 | 2.7 |
| Geronimo | 3.8 | 50.7 | 2.0 | 1.9 | 1.6 |
| HB 329 | 4.8 | 61.1 | 2.9 | 3.1 | 2.9 |
| HB 130 | 4.0 | 46.7 | 1.9 | 1.9 | 2.1 |
| LSD (P = 0.05) | 0.8 | 19.3 | 0.9 | 0.7 | 0.4 |
| CV | 10.7 | 15.8 | 16.2 | 12.2 | 19.6 |

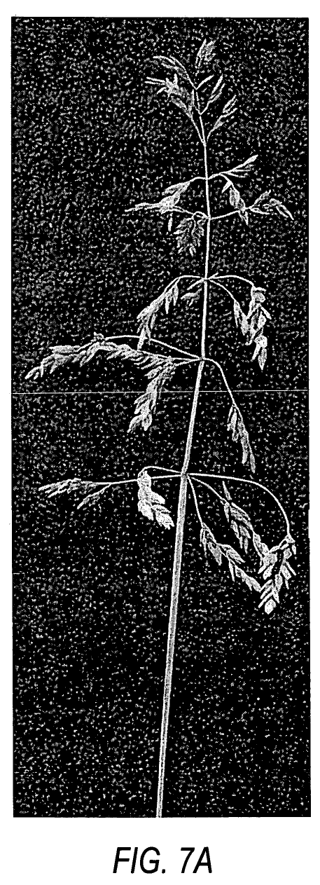

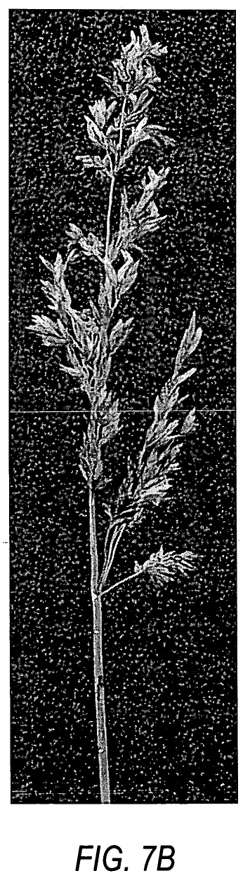

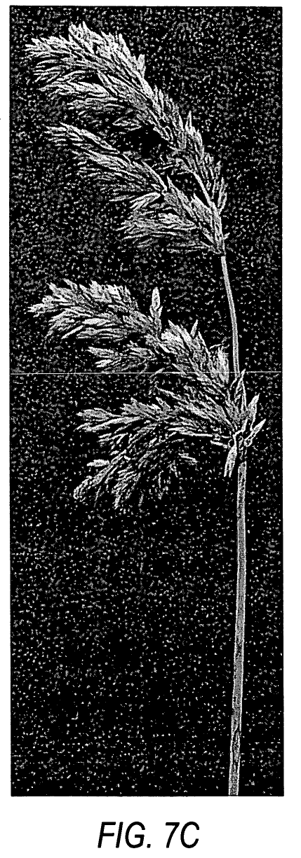

Table 7 shows the percent rating of panicle type of various bluegrass cultivars grown in a field nursery at Gervais, Ore. Data were collected from three replicates. HB 129, HB 130, HB 329 and HB342 all show greater panicle openness than Reveille hybrid bluegrass. See also FIGS. 7A-C.

TABLE 7
Three replicates with 15 subsamples per replicate

| | Panicle Type | | |
|---|---|---|---|
| | Open | Intermediate | Compact |
| Plant | % | % | % |
| HB 129 | 96.8 | 3.2 | 0.0 |
| HB 342 | 100.0 | 0.0 | 0.0 |
| HB 130 | 100.0 | 0.0 | 0.0 |
| HB 329 | 92.6 | 7.4 | 0.0 |
| Reveille | 27.0 | 59.8 | 13.2 |
| Geronimo | 100.0 | 0.0 | 0.0 |
| Ascot | 98.3 | 1.7 | 0.0 |
| Midnight | 100.0 | 0.0 | 0.0 |
| Buckingham | 100.0 | 0.0 | 0.0 |
| HB 13 | 53.4 | 46.6 | 0.0 |
| HB 96 | 71.4 | 23.9 | 4.7 |
| HB 99 | 62.8 | 29.8 | 7.3 |
| HB 101 | 56.7 | 43.0 | 0.0 |
| Poa arachnifera TX 51-90 | 0.0 | 25.0 | 75.0 |
| Poa arachnifera TX 39-88 | 0.0 | 15.4 | 84.7 |
| Poa arachnifera TX 49-90 | 0.0 | 25.7 | 74.3 |
| Kelly | 98.4 | 1.6 | 0.0 |
| LSD (P = .05) | 12.8 | 15.4 | 10.1 |
| CV | 10.2 | 69.6 | 56.9 |

Table 8 shows the rate of turf establishment. Establishment of hybrid bluegrasses and Kentucky bluegrasses were established in turf based upon visual rating for plot coverage. All seeds were obtained from commercial or breeder seed lots. HB 129 was comparable with the best Kentucky bluegrasses. HB 342 was slower in establishment. All varieties are established from commercial or in the case of HB 342, breeders seed. HB 329 is related to Ascot Kentucky bluegrass, and this hybrid bluegrass demonstrates an emergence more similar to that parental line than to Texas bluegrass.

Table 9 shows seedlings emergence in field at Gervais, Ore. which included HB 129 and a Texas Bluegrass×Kentucky bluegrass hybrid, Reveille. As indicated, the emergence of this variety was similar to Kentucky bluegrass. This data indicates that HB 129 has the Kentucky bluegrass seed physiology characteristics. It is noted that the emergence is not related to germination rate, the Texas bluegrass×Kentucky bluegrass hybrid, Reveille, does not behave in a similar fashion as Kentucky bluegrass.

TABLE 8
Average Percent establishment in turf plots-seeded at 2 pounds per 1000 sq.
feet. (Seeding rate) (4 replicates)

| | Days | | | | | | | | | 3 | 4 |
|---|---|---|---|---|---|---|---|---|---|---|---|
| Plant | 11 | 14 | 18 | 21 | 28 | 35 | 42 | 49 | 63 | mons | mons |
| HB 129 | 55 | 75 | 75 | 90 | 92 | 95 | 93 | 98 | 99 | 97 | 98 |
| HB 342 | 33 | 57 | 63 | 65 | 82 | 85 | 80 | 88 | 91 | 91 | 95 |
| South Dakota | 55 | 77 | 77 | 78 | 90 | 93 | 92 | 97 | 96 | 93 | 96 |
| Abbey | 48 | 73 | 75 | 73 | 85 | 92 | 93 | 97 | 97 | 97 | 97 |
| Midnight | 43 | 63 | 67 | 68 | 82 | 87 | 83 | 92 | 94 | 94 | 95 |
| Ascot | 42 | 62 | 67 | 73 | 82 | 87 | 83 | 95 | 93 | 93 | 94 |
| Geronimo | 40 | 60 | 70 | 72 | 82 | 82 | 83 | 96 | 96 | 96 | 98 |
| Touchdown | 37 | 50 | 53 | 55 | 67 | 72 | 70 | 75 | 79 | 79 | 80 |
| Reveille | 16 | 28 | 28 | 32 | 42 | 48 | 50 | 53 | 53 | 59 | 59 |
| LSD (P = 0.05) | 29.6 | 39.6 | 38.8 | 35.5 | 32.0 | 33.1 | 34.2 | 35.2 | 33.1 | 28.3 | 28.2 |
| CV | 41.6 | 37.8 | 35.1 | 30.9 | 23.7 | 23.3 | 24.4 | 23.2 | 21.6 | 18.4 | 18.0 |

TABLE 9
Average Number of seeds emerged out of 200 seeds planted per 2′ × 2′
plot (trial included 4 replicates)

| | Days | | | | | | |
|---|---|---|---|---|---|---|---|
| Plant | 14 | 21 | 28 | 42 | 56 | 70 | 84 |
| HB 129 | 106.3 | 125.8 | 130.8 | 132.5 | 133.5 | 135.0 | 135.5 |
| South Dakota | 130.0 | 143.5 | 147.0 | 149.3 | 149.8 | 151.0 | 151.3 |
| Abbey | 115.3 | 129.8 | 133.0 | 135.0 | 136.3 | 136.5 | 137.5 |
| Touchdown | 108.8 | 119.0 | 123.8 | 124.5 | 126.0 | 126.3 | 126.3 |
| Midnight | 94.5 | 108.3 | 114.0 | 117.5 | 118.0 | 119.0 | 119.5 |
| Ascot | 77.5 | 102.8 | 112.3 | 117.3 | 120.3 | 121.3 | 122.0 |
| HB 329 | 59.8 | 83.0 | 88.8 | 93.0 | 95.8 | 98.0 | 98.5 |
| Texas | 5.0 | 6.3 | 7.0 | 7.5 | 7.8 | 8.0 | 8.3 |
| bluegrass | | | | | | | |
| LSD (P = 0.05) | 18.2 | 21.6 | 21.4 | 20.8 | 20.3 | 20.1 | 19.0 |
| CV | 13.5 | 13.8 | 13.1 | 12.5 | 12.1 | 11.9 | 11.2 |

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

What is claimed:

1. A hybrid plant derived from crossing Poa arachnifera and Poa pratensis grasses as plant parents, the hybrid plant or its seed exhibiting at least one characteristic selected from the group consisting of:
(i) a reduced amount of cotton webbing on a seed of the hybrid plant;
(ii) a reduced cotton webbing density on a seed of the hybrid plant;
(iii) a reduced length of cotton webbing as a percentage of lemma length on a seed of the hybrid plant;
(iv) increased panicle openness; and
(v) reduced panicle compactness.

2. The hybrid plant of claim 1, wherein the reduced amount of cotton webbing on the seed of the hybrid plant; the reduced cotton webbing density on the seed of the hybrid plant; the reduced length of cotton webbing as a percentage of lemma length on the seed of the hybrid plant; the increased panicle openness; and the reduced panicle compactness, are in comparison with Reveille.

3. The hybrid plant of claim 1 wherein one of the plant parent is Poa arachnifera Torr.

4. The hybrid plant of claim 3 wherein the Poa arachnifera Torr. is a female plant.

5. The hybrid plant of claim 1 wherein one of the plant parent is Poa pratensis L.

6. The hybrid plant of claim 1 wherein the plant parents are Poa arachnifera Torr. and Poa pratensis L.

7. The hybrid plant of claim 1 wherein the amount of cotton webbing on a seed of the hybrid plant is rated 2 or below.

8. The hybrid plant of claim 1 wherein the cotton webbing density on a seed of the hybrid plant is rated 2.6 or below.

9. The hybrid plant of claim 1 wherein the length of cotton webbing as a percentage of lemma length is less than about 75%.

10. The hybrid plant of claim 1 wherein the panicle openness is in a range of about 30% to about 100%.

11. The hybrid plant of claim 1 wherein the panicle compactness is less than about 5%.

12. The hybrid plant of claim 1 wherein the reduced amount of cotton webbing on a seed is determined after conditioning of the seed.

13. The hybrid plant of claim 1, wherein the hybrid plant is derived from backcrossing to a Poa arachnifera.

14. The hybrid plant of claim 1, wherein the hybrid plant is derived from backcrossing to a Poa pratensis.

15. The hybrid plant of claim 1, wherein the hybrid plant is derived from intercrossing to a Poa arachnifera and a Poa pratensis hybrid plant.

16. The hybrid plant of claim 1, wherein the hybrid plant is in a sod.

17. The hybrid plant of claim 1, wherein the hybrid plant is in a golf course fairway.

18. The hybrid plant of claim 1, wherein the hybrid plant is in a golf course rough.

19. The hybrid plant of claim 1, wherein the hybrid plant is in a lawn.

20. The hybrid plant of claim 1, wherein the hybrid plant is in an athletic field.

21. The hybrid plant of claim 1, wherein the hybrid plant is in a park.

22. The hybrid plant of claim 1, wherein the hybrid plant is in a hay stand.

23. The hybrid plant of claim 1, wherein the hybrid plant is in a pasture stand.

24. The hybrid plant of claim 1, wherein the hybrid plant is in a roadside or right-of way.

25. A progeny of a hybrid plant according to claim 1.

26. A seed of the hybrid plant according to claim 1.

27. A seed mixture containing the seed of claim 26.

28. A Poa arachnifera and Poa pratensis hybrid plant grown from seed deposited on Jul. 22, 2005 under American Type Culture Collection (ATCC) No ______.

29. A progeny of the hybrid plant according to claim 28.

30. The progeny of claim 29 wherein the hybrid plant is backcrossed to a Poa arachnifera.

31. The progeny of claim 30 wherein the hybrid plant is backcrossed to a Poa arachnifera Torr.

32. The progeny of claim 29 wherein the hybrid plant is backcrossed to a Poa pratensis.

33. The progeny of claim 32 wherein the hybrid plant is backcrossed to a Poa pratensis L.

34. The progeny of claim 29 wherein a hybrid plant of Poa arachnifera Torr. and Poa pratensis L. is intercrossed with a hybrid plant of Poa arachnifera and Poa pratensis.

35. The progeny of claim 34 wherein a hybrid plant of Poa arachnifera Torr. and Poa pratensis L. is intercrossed with a hybrid plant of Poa arachnifera Torr. and Poa pratensis L.

36. A seed of the hybrid plant according to claim 28.

37. A seed mixture containing the seed of claim 36.

38. A seed deposited on Jul. 22, 2005 under American Type Culture Collection (ATCC) No ______.

39. A plant part of the hybrid plant of claim 1, wherein the plant part is a sprig, leaf, pollen, embryo, root, root tip, anther, flower, cotton, lemmas, spikelet, floret, rhizome, node, stem, tiller or culture thereof.

40. A method of producing a seed of a hybrid plant comprising
a) planting the seed according to claim 26 under conditions that result in the germination of the seed, growth of the hybrid plant and setting of progeny seed; and;
b) harvesting of the progeny seed.

41. A method of producing progeny of the hybrid plant of claim 1 comprising:
a) crossing Poa arachnifera Torr. and Poa pratensis L.;
b) harvesting the seed; and
c) germinating the seed to produce at least one progeny of the hybrid plant.

42. The method of claim 41 wherein the hybrid plant is backcrossed to a Poa arachnifera.

43. The method of claim 42 wherein the hybrid plant is backcrossed to a Poa arachnifera Torr.

44. The method of claim 41 wherein the hybrid plant is backcrossed to a Poa pratensis.

45. The method of claim 44 wherein the hybrid plant is backcrossed to a Poa pratensis L.

46. The method of claim 41 wherein the hybrid plant is intercrossed with a hybrid of Poa arachnifera and Poa pratensis.

47. The method of claim 46 wherein the hybrid plant is intercrossed with a hybrid of Poa arachnifera Torr. and Poa pratensis L.

48. A method of producing a progeny of the hybrid plant of claim 1 comprising the steps of:
a) selecting a Poa arachnifera Torr. and a Poa pratensis L. grass plant;
b) crossing the plants of step a);
c) harvesting and growing the seed resulting from the crossing in step b);
d) selecting F₁ plants grown from the seed of step c);
e) harvesting seed from the F₁ plants;
f) selecting seed with reduced amount or density of webbing on the seed; and
g) germinating the seed to produce the hybrid plant.

49. The method of claim 48 further comprising the step of selecting for F₂ plants with a rapid germination time.

50. The method of claim 48 further comprising the step of growing and selecting for high levels of apomixis and/or commercially acceptable turfgrass quality characteristics.

51. The method of claim 48 wherein the Poa arachnifera Torr. is a female plant.

52. The method of claim 48 wherein the Poa arachnifera Torr. is selected for a superior turfgrass characteristic.

53. The method of claim 48 wherein the Poa pratensis L. is selected for a superior seed yield characteristic.

54. The method of claim 48 wherein the F₁ plants selected in step d) comprises perfect flowers.

55. The method of claim 48 wherein the hybrid plant is backcrossed to a Poa arachnifera.

56. The method of claim 55 wherein the hybrid plant is backcrossed to a Poa arachnifera Torr.

57. The method of claim 48 wherein the hybrid plant is backcrossed to a Poa pratensis.

58. The method of claim 57 wherein the hybrid plant is backcrossed to a Poa pratensis L.

59. The method of claim 48 wherein the hybrid plant is intercrossed with a hybrid of Poa arachnifera and Poa pratensis.

60. The method of claim 59 wherein the hybrid plant is intercrossed with a hybrid of Poa arachnifera Torr. and Poa pratensis L.
